(19) United States
(12) Patent Application Publication
(10) Pub. No.: US 2025/0127950 A1
(43) Pub. Date: Apr. 24, 2025

(54) Air Conditioning Disinfection and Sterilization Device

(71) Applicant: Fresh On Limited, Kowloon (HK)

(72) Inventor: Ching Man MOK, Kowloon (HK)

(21) Appl. No.: 18/489,530

(22) Filed: Oct. 18, 2023

(51) Int. Cl. A61L 9/20; F24F 8/167; F24F 8/22

(52) U.S. Cl. CPC A61L 9/205; F24F 8/167; F24F 8/22; A61L 2209/12; A61L 2209/16

(57) ABSTRACT

An air conditioning disinfection and sterilization device can be detachably installed at an air intake of an air conditioner and cover the air intake. The device includes a housing with a disinfection and sterilization unit centrally arranged in the housing. The sidewall of the housing is uniformly distributed with air inlets along the radial direction, and the housing is also equipped with an air outlet corresponding to the air intake of the air conditioner. This device can be conveniently fixed to the air intake of the air conditioner using a bracket. Under the negative pressure generated by the air intake of the air conditioner, external air is drawn into the housing through air inlets, and then the disinfection and sterilization unit within the housing eliminates bacteria or viruses in the air.

### TECHNICAL FIELD

This invention relates to a disinfection device, in particular, to a device installed in an air conditioner for disinfecting and sterilizing air.

### BACKGROUND

Air conditioner is the most commonly used temperature regulation device. It works by drawing external air into the interior of the air conditioner and passing it through internal heat exchange fins to cool or heat the air, thus adjusting the air temperature. To enhance energy efficiency during air conditioning, it often draws indoor air for cooling or heating before releasing it back indoors. Consequently, the air circulation between the indoor space with air conditioning and the outdoor environment is limited. In densely populated spaces with bacteria or viruses, air circulation through the air conditioning system could potentially spread these microorganisms throughout the entire area. Although some air conditioning systems are equipped with filters or disinfection devices like silver ions, these devices have certain lifespans or capacities, requiring regular replacement. Moreover, replacing or cleaning these devices can be quite cumbersome.

### SUMMARY

In response to the shortcomings of the existing technology mentioned above, the present invention provides an air conditioning disinfection and sterilization cover device. This device can be directly installed at an air intake of existing air conditioners to achieve disinfection and sterilization functions. Importantly, it does not interfere with the normal operation of air conditioners and is simple to install.

The present invention is realized through the following technical solution: An air conditioning disinfection and sterilization cover device, which can be detachably installed at an air intake of an air conditioner and cover the air intake. The device comprises a housing, wherein a disinfection and sterilization unit is centrally arranged in the housing. The sidewall of the housing is uniformly distributed with air inlets along its radial direction. The housing is also equipped with an air outlet, which corresponds to the air intake of the air conditioner. Negative pressure is formed at the air intake of the air conditioner, allowing air outside the air inlets to enter the housing and pass through the disinfection and sterilization unit for disinfection and sterilization before being introduced into the air intake of the air conditioner.

Preferably, the air inlets form a spiral air passage extending from the sidewall of the housing toward the center of the housing. The air passes through the spiral air passage and forms an air vortex at the central position of the housing where the disinfection and sterilization unit is located.

Preferably, the disinfection and sterilization unit is an ultraviolet lamp unit.

Preferably, the ultraviolet lamp unit is an ultraviolet LED.

Preferably, there is a transparent window at the center of the housing to display the working status of the ultraviolet lamp unit.

Preferably, the distance between the ultraviolet LED and the bottom surface of the perforated plate is 5-100 mm.

Preferably, the air outlet of the housing is covered with a perforated plate.

Preferably, the outer surface of the perforated plate is coated with a photocatalytic layer for disinfection and sterilization.

Compared to the prior art, the advantageous effect of the present invention is that the device can be conveniently fixed and installed at the air intake of the air conditioner using a bracket. Under the negative pressure generated by the air intake of the air conditioner, external air is drawn into the housing. The disinfection and sterilization unit inside the housing eliminates bacteria or viruses in the air, and the treated air is then introduced into the air conditioner for cooling or heating through the air intake. This effectively prevents the potential spread of bacteria or viruses through the air conditioning system.

### DESCRIPTION OF DRAWINGS

To provide a clearer understanding of the technical solution in the embodiments of the present invention, the following description is given in conjunction with the drawings.

FIG. 1 is a perspective view of the present invention.

FIG. 2 is an exploded view of the present invention.

### EMBODIMENTS

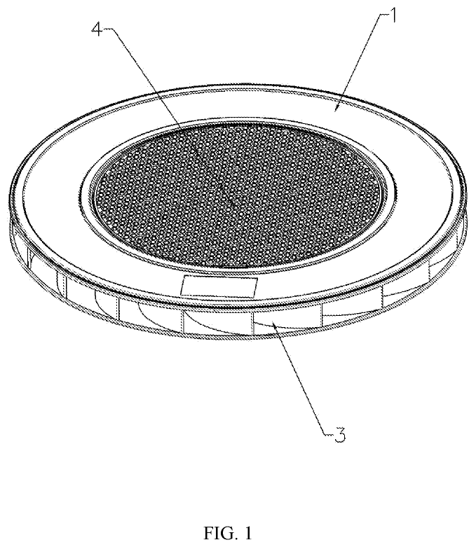

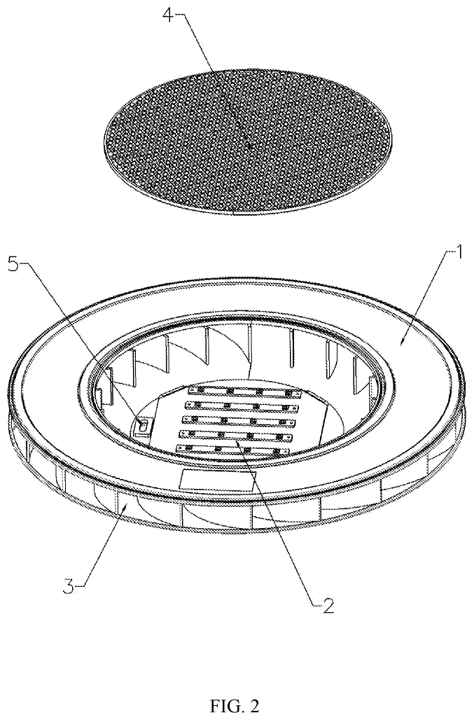

With reference to FIG. 1 and FIG. 2, an air conditioning disinfection and sterilization device is described as follows: The device can be detachably installed at an air intake of an air conditioner and cover the air intake. It comprises a housing (1) with a disinfection and sterilization unit (2) centrally arranged in the housing (1). The sidewall of the housing (1) is uniformly distributed with air inlets (3) along the radial direction, and the housing (1) is also equipped with an air outlet corresponding to the air intake of the air conditioner. This device can be conveniently fixed to the air intake of the air conditioner using a bracket. Under the negative pressure generated by the air intake of the air conditioner, external air is drawn into the housing (1) through the air inlets (3), and then the disinfection and sterilization unit (2) within the housing (1) eliminates bacteria or viruses in the air. The treated air then enters the air conditioner through the air intake for cooling or heating, effectively preventing the potential spread of bacteria or viruses through the air conditioning system.

Due to the overall slim design of this device, and considering that the disinfection and sterilization unit (2) requires some time to complete the disinfection and sterilization process, it's necessary to extend the residence time of the air within the housing (1). This will allow the disinfection and sterilization unit (2) to more effectively treat the inhaled air for disinfection and sterilization. Therefore, the air inlets (3) are designed as a spiral air passage extending from the sidewall of the housing (1) towards the center of the housing (1). After passing through the spiral air passage, the air forms an air vortex at the center of the housing (1) where the disinfection and sterilization unit (2) is located. The air vortex flows around the disinfection and sterilization unit (2), ensuring thorough contact with the disinfection and sterilization unit (2) and completing the disinfection and sterilization process. Additionally, the generated air vortex increases the residence time of the air within the housing (1), meeting the requirements for effective disinfection and sterilization.

The aforementioned disinfection and sterilization unit (2) can be realized using various methods, such as ozone generators, silver ions, and others. However, these methods are not very suitable for air conditioning systems. For instance, ozone can generate unpleasant odors and cause irritation to the respiratory system. Silver ions also have a limited lifespan and require regular replacement, making them less practical. In contrast, this invention preferably utilizes ultraviolet (UV) lamps as the sterilization and disinfection unit. UV lamps with appropriate wavelengths can achieve sterilization and disinfection effects without producing ozone or minimizing ozone generation. Moreover, the ultraviolet rays produced by UV lamps can effectively cover the entire space within the housing (1), enhancing the efficiency of sterilization and disinfection while being energy-efficient. To further enhance energy efficiency and reduce the device's size, ultraviolet LED lights are chosen for the UV lamp unit. LEDs require very little power; as low as 10 mW is sufficient for sterilization and disinfection purposes. Different models of ultraviolet LED lights have varying effects on sterilization and disinfection distance. The invention's testing has shown that a distance of 5-100 mm between the ultraviolet LED lights and the bottom surface of the perforated plate achieves the optimal effect. This approach also contributes to reducing the overall size of the device.

For the convenience of users to easily understand the device's operational status, there is a transparent window (5) installed in the center of the housing (1) to display the working status of the ultraviolet lamp unit. A small amount of ultraviolet light passes through the transparent window (5), allowing users to easily determine whether the device is in operation simply by observing the transparent window (5).

Furthermore, a perforated plate (4) is positioned at the air outlet of the housing (1). The perforated plate (4) helps maintain the air vortex formed inside the housing (1), thus increasing the residence time of the air within the housing (1). Additionally, the outer surface of the perforated plate (4) is coated with a photocatalytic layer designed for disinfection and sterilization. Under the illumination of the ultraviolet lamp unit, the photocatalytic layer generates strong disinfection and sterilization effects. This enhances the air disinfection and sterilization process as air passes through the perforated plate (4).

1. An air conditioning disinfection and sterilization device, wherein: the device is detachably installed at an air intake of an air conditioner and covers the air intake, comprising a housing, wherein the disinfection and sterilization unit is centrally arranged in the housing, and the sidewall of the housing is uniformly distributed with air inlets along the radial direction; the housing is also provided with an air outlet, which corresponds to the air intake of the air conditioner; the air intake of the air conditioner forms negative pressure, causing air outside the air inlets to enter the housing and pass through the disinfection and sterilization unit to complete disinfection and sterilization before entering the air intake of the air conditioner.

2. The air conditioning disinfection and sterilization device according to claim 1, wherein: the air inlets form a spiral air passage extending from the sidewall of the housing towards the center of the housing; after passing through the spiral air passage, the air forms an air vortex at the central position of the housing where the disinfection and sterilization unit is located.

3. The air conditioning disinfection and sterilization device according to claim 1, wherein: the disinfection and sterilization unit is an ultraviolet lamp unit.

4. The air conditioning disinfection and sterilization device according to claim 3, wherein: the ultraviolet lamp unit is an ultraviolet LED.

5. The air conditioning disinfection and sterilization device according to claim 3, wherein: a transparent window for displaying the working status of the ultraviolet lamp unit is provided at the center of the housing.

6. The air conditioning disinfection and sterilization device according to claim 1, wherein: a perforated plate is covered at the air outlet of the housing.

7. The air conditioning disinfection and sterilization device according to claim 6, wherein: the distance between the ultraviolet LED and the bottom surface of the perforated plate is 5-100 mm.

8. The air conditioning disinfection and sterilization device according to claim 6, wherein: the outer surface of the perforated plate is coated with a photocatalytic coating for disinfection and sterilization.
